(19) United States
(12) Patent Application Publication
(10) Pub. No.: US 2019/0071626 A1
(43) Pub. Date: Mar. 7, 2019

(54) INTEGRATED DEVICE FOR PRODUCING ELECTRICITY, BIODIESEL, HYDROXY METHYL FURFURAL HMF, AND CHAR COAL FROM WASTE (SEWAGE, DOMESTIC DISPOSALS, AGRICULTURAL WASTES)

(71) Applicants: Osayed Sayed M. Abu-Elyazeed, Ramadan City, Ashargya (EG); Bader Mousa Ahmed Alsalahi, Makah (SA); Abdullah Mohamed A. Bin Salamah, (US); Omar Mohamed Sadek, EI Segundo, CA (US)

(72) Inventors: Osayed Sayed M. Abu-Elyazeed, Ramadan City, Ashargya (EG); Bader Mousa Ahmed Alsalahi, Makah (SA); Abdullah Mohamed A. Bin Salamah, (US); Omar Mohamed Sadek, EI Segundo, CA (US)

(21) Appl. No.: 16/067,300

(22) PCT Filed: Dec. 30, 2016
(86) PCT No.: PCT/IB2016/058125

§ 371 Date: Nov. 20, 2018

(30) Foreign Application Priority Data
Dec. 30, 2015 (GC) 2015-30684 30

(51) Int. Cl. C12M 1/107; C12M 3/00; C12M 1/00

(52) U.S. Cl. CPC C12M 21/04; C12M 43/08; C12M 23/44; C12M 23/36

(57) ABSTRACT

A device for producing energy from waste including a power generator, a digester, and .a generator utilizing biodiesel, active coal, tar, and ashes. The latter includes a purification chamber, a gas generator, a radiator, a compressor, and a reactor. The device includes an HMF producer comprising a primary treatment tank, a pump, a reactor, an extractor and a purification tank. The power generator may be at least one of a diesel motor, a gas turbine and steam cycle. The digester is structured to produce biogas.

### RELATED APPLICATIONS

This application claims the benefit under 35 U.S.C. § 119(e) of Gulf Coast patent application Ser. No. GC 2015-30684 filed on Dec. 30, 2015 and titled Integrated Device for Producing Electricity, Biodiesel, Hydroxy Methyl Furfural HMF, and Char Coal from Waste (Sewage, Domestic Disposals, Agricultural Wastes), and is related to PCT patent application serial no. PCT/IB2016/058125 filed on Dec. 30, 2016, also titled Integrated Device for Producing Electricity, Biodiesel, Hydroxy Methyl Furfural HMF, and Char Coal from Waste (Sewage, Domestic Disposals, Agricultural Wastes) the entire content of which, is incorporated herein by reference except to the extent that disclosure therein is inconsistent with the disclosure herein.

### FIELD OF THE INVENTION

This invention relates to an integrated process of generating diverse types of energy using different types of wastes.

### BACKGROUND OF THE INVENTION

Presently, the energy crisis is well-known to be caused by the lack of energy resources, such as petroleum oil, due to the decrease in production and increase in consumption. Additionally, the environmental impact of oil, gas and coal combustion causes a crisis known as the greenhouse effect. The usual methods of waste disposal including sewage, household and agricultural waste disposal intensifies this environmental catastrophe.

It is noteworthy that electricity is generated using thermal devices without hot exhausts estimated at 30% of the thermal content of the used fuel. Accordingly, these devices, such as diesel motors and gas turbines can play a significant role in overcoming the energy and environmental crisis.

Waste, also known as “biomass”, can also be classified into two types. The first type is recyclable waste such as paper, plastic material and metal that are reprocessed to produce useful products. The second type of waste is called unacceptable waste, such as sewage, food residues and agricultural wastes and is divided into volatile and nonvolatile substances and ashes.

This type of biomass can be used as a source of energy using one or more of the three following methods: 1. anaerobic digestion, 2. thermal conversion to gases and 3. thermochemical conversion. As such, any of the abovementioned methods can be either endothermic (such as anaerobic digestion and thermochemical conversion) or exothermic (such as thermal conversion to gases). In commonly known devices these methods can be used separately without any linkage among them or any linkage to power generation devices.

However, there is a serious lack of integration between biomass conversion devices and power generation devices. In addition, there is a lack of knowledge and technology in the field of thermochemical conversion of agricultural waste. This is true in the field of purification of different products such as synthetic gases (syngas) and hydroxymethylfurfural (HMF), which has not been resolved until the present invention.

### SUMMARY OF THE INVENTION

The present invention uses the exhausts or outputs of devices to feed one or a plurality of the other devices while purifying HMF and syngas with natural and inexpensive materials. One aspect of the invention utilizes an electrical power generator with a diesel motor, gas turbine or steam cycle as a primary generator with hot gases as exhausts. Another aspect of the invention is an anaerobic digester for producing biogas with a carbon-rich material as an exhaust of the endothermic process. Yet another aspect of the invention is a system for converting biomass to gases for production of syngas, biodiesel, tar, charcoal and ashes with heat as an exhaust of the exothermic process. The final aspect of the invention is a thermochemical converter for producing HMF with non-convertible solids as an exhaust of the endothermic process.

The invention integrates these different aspects. In addition, this invention requires complex connections and a control system for the devices in order to ensure integration among them. The invention requires actual data resulting from practical experiences in different types of conditions such as flow rate, reaction time, temperature, pressure, catalyst type and pH.

The devices are low-energy density devices (generated energy/consumed energy) because of the use of external energy resources (heat and electricity, etc.) as is the case in exhausts (substances and heat, etc.). The present invention is known as a high-energy density device consisting of four zero-emission parts. The zero-emission is a result utilizing exhausts as energy or as an input source for the other aspects of the device.

Accordingly, the inputs to the first part, electrical power generator, are air and fuel (the biogas from the second part, the anaerobic digester, a percentage of the syngas produced from the third part of the invention and the gas generator from biomass). Furthermore, the outputs of this first part are electrical power as a final product. Additionally, high-temperature exhaust gases are used as a source of heat and partially oxygen-containing gases for the third part of the invention to complete the conversion to gases.

The inputs to the second part of this invention, the anaerobic digester, are sewage (wastes), heat (generated from the syngas produced from the third part, the system of conversion of biomass to gases during the process of purifying the gases), anaerobic bacteria (from soil) and an acidic solution added to adjust the pH of the solution in the digestion area. The outputs from the second part are biogas (used as a fuel for the first part power generator) and carbon-rich materials (used as a fertilizer in unpolluted drainage or a source of biomass for the third part of the invention).

The third part of the invention includes the system of converting biomass to gases. This may be biomass (household wastes, carbon-rich materials produced from the second part of the device and solids produced from the fourth part of the invention, thermochemical conversion of agricultural wastes), oxygen-containing gases (generated from the exhaust of the first part of the device, power generator, and the air), and a heat source (from the hot exhaust gases generated from the first). The outputs from the third part may include heat (used to increase the temperature of the second part and the hydrolysis processing of agricultural wastes in the fourth part of the invention), syngas, a hydrogen-rich gas purified in chambers around the digester containing dolomite, eggshell, charcoal, ashes and sands as catalysts. About half of these gases are used as fuel for the first part, while the other half is converted into biodiesel. This happens during the Fischer-Tropsch process of compressing syngas as a final second product of the invention with silicon carbide, cobalt and calcium as catalysts. This has a shelf life of five years. Tar is a final third product produced during the syngas-cooling process. Syngas compression and charcoal are the final fourth products produced from converting the biomass to gases. This is the case with ashes, the final fifth product.

The fourth pail of the invention is a thermochemical device used to treat agricultural wastes and comprises three phases. The first stage is a hydrolysis process that represents primary treatment of agricultural waste as cellulose materials. Inputs are water (used for hydrolysis of these wastes by converting cellulose to glucose with hydrochloric acid of a volume of 3%) and heat (generated from the third part during syngas cooling and before syngas compression to meet the requirements for the Fischer-Tropsch process). The second stage is the dehydration process applied as a final treatment of glucose and converting it to fructose and then to hydroxymethylfurfural (RMF). The inputs of the final sixth product of the invention are heat (generated from the third part during the Fischer-Tropsch process of hot and compressed syngas, with DMSO as solvent and chromium chloride as catalyst). The third stage is a process for purifying the HMF produced from the previous stage and recovering DMSO as a reusable solvent. This uses charcoal to elute DMSO, as a polarity material, and collects HMF. Afterwards, the coal-heating process starts to evaporate eluted DMSO and vapor is condensed to recover DMSO. The remaining products of the fourth part are HMF and the remaining non-cellulose solids. This is used as a source of biomass for the third part of this device, the system of conversion of biomass to gases.

### BRIEF DESCRIPTION OF THE DRAWINGS

FIG. 1 is a perspective view of one embodiment of the invention illustrating four integrated aspects.

FIG. 2 is an exploded view of a digester illustrated in FIG. 1 indicates the unique design of digester with purification chamber of syngas.

FIG. 3 is a flowchart illustrating the flow of material to and from each component of the invention illustrated in FIG. 1.

### DETAILED DESCRIPTION OF THE INVENTION

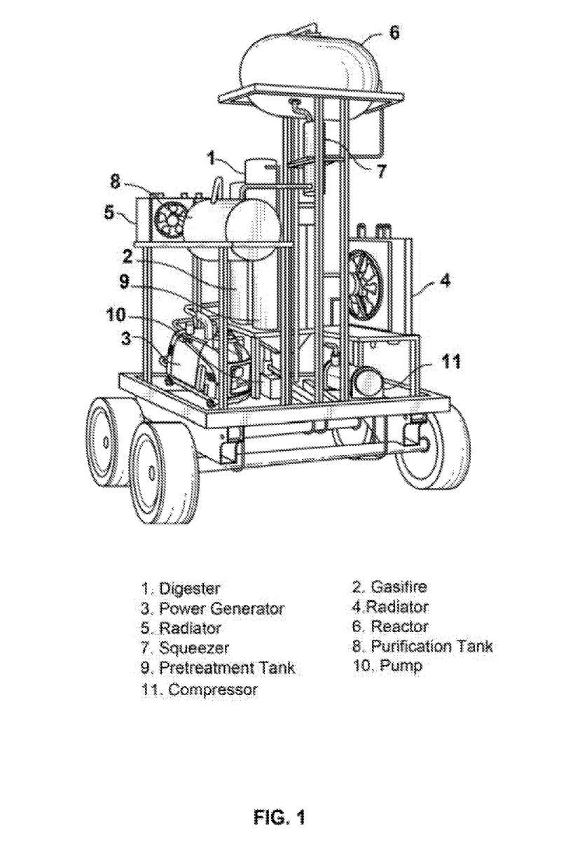

Referring to FIGS. 1-3, Digester 1 contains sewage within circumstances of diluted acid and temperature of 35-45° C. of anaerobic digestion and the production of biogas after a digestion period of 15-20 days. This can be used as fuel for power generator 3. The power generator 3 produces electrical power being the first product of the device, and also produces exhaust that is used to provide gas generator 2 with heat and gases with oxygen as indicated in FIG. 1. Gas generator 2 is provided with household waste and the rest of the digestion process, as well as the rest of the agricultural waste treatment process and what is obtained from extractor 7. Gas generator 2 Gas produces active coal and ashes representing the second and third products of this invention, and also produces syngas. Syngas is purified in chambers surrounding digester 1 for heating purposes, as such chambers contain the dolomite, eggshell, charcoal, ashes, sand to separate tar being the fourth product of this device. Purified syngas is cooled in two phases, the first of which is done in primary treatment tank 9 to convert the agricultural cellulosic waste into glucose during the hydrolysis for four hours in diluted hydrochloric acid solution at 3%. The second phase of syngas cooling is done in radiator 4. About half of the syngas after cooling is provided to power generator 3, while the rest is compressed to 25 bar, heated to 300° C. through compressor 11 and then moved to reactor 6 in presence of silicon carbide-calcium-cobalt as a catalyst for the conversion of syngas into biodiesel being the fifth product of this device. The glucose produced during the hydrolysis of agricultural cellulosic waste is melted by adding DMSO to this glucose in primary treatment tank 9 and mixing this mixture for an hour in presence of chromic chloride to convert this glucose into fructose. This mixture is composed of DMSO, glucose, fructose and chromic chloride. The unconverted biomass is pumped through primary treatment tank 9 through pump 10 to reactor 6 for the production of HMF being the sixth product by heating this mixture at 120° C. or 250° C. for two hours or fifteen minutes, respectively, through the use of the above-mentioned compressed syngas. The new resulted product, which is HMF, DMSO, chromic chloride and unconverted biomass, is first poured into extractor 7 to separate the remaining biomass and then supply the residue to gas generator 2, and second to purification tank 8 filled with active coal to adsorb the solvent DMSO and extract the purified HMF being the sixth product of this invention.

Also, purification tank 8 is heated to 192° C. to evaporate the solvent DMSO, then this steam is condensed in radiator 5.

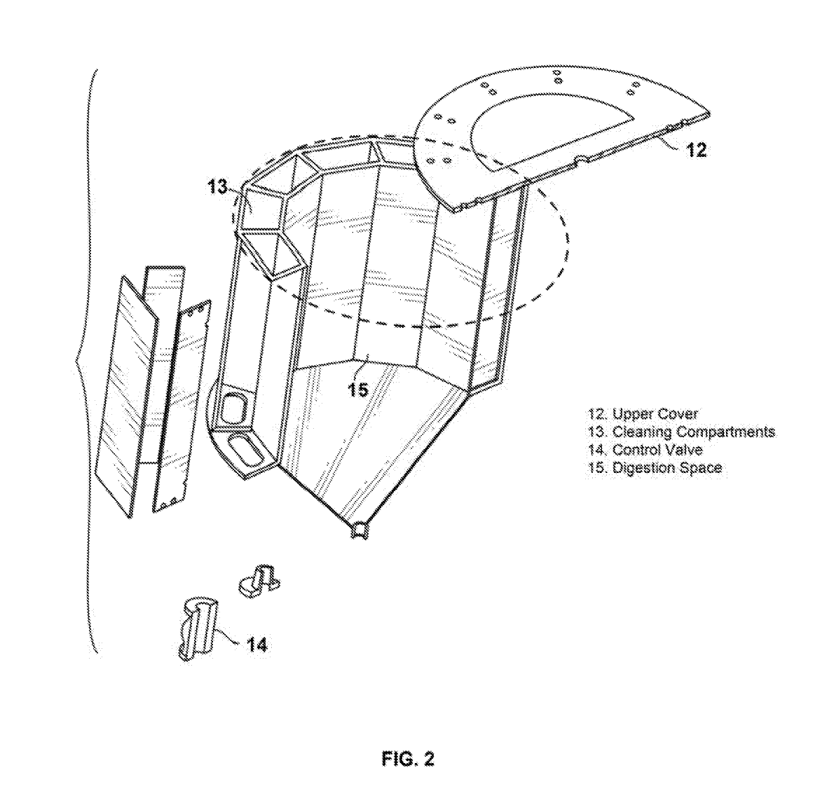

With reference to FIG. 2, there are 3D details of the unique design of digester 1. This design is based on the execution of the principle of downsizing this part. It contains upper cover 12, which can be used as cover of chamber 13 and digestion area 15. It is also equipped with holes for refilling with sewage and removal of produced biogas, holes for measurement and security and a tool to flip sewage inside the digester. Chamber 13 is arranged to form digestion area 15 and is considered as cleaning passages of syngas filled with dolomite, egg shell, active coal, ashes and sand as catalytic agents. Syngas is also used to heat up digestion area 15. The syngas, together with the remaining part after digestion, can be removed through control valves 14.

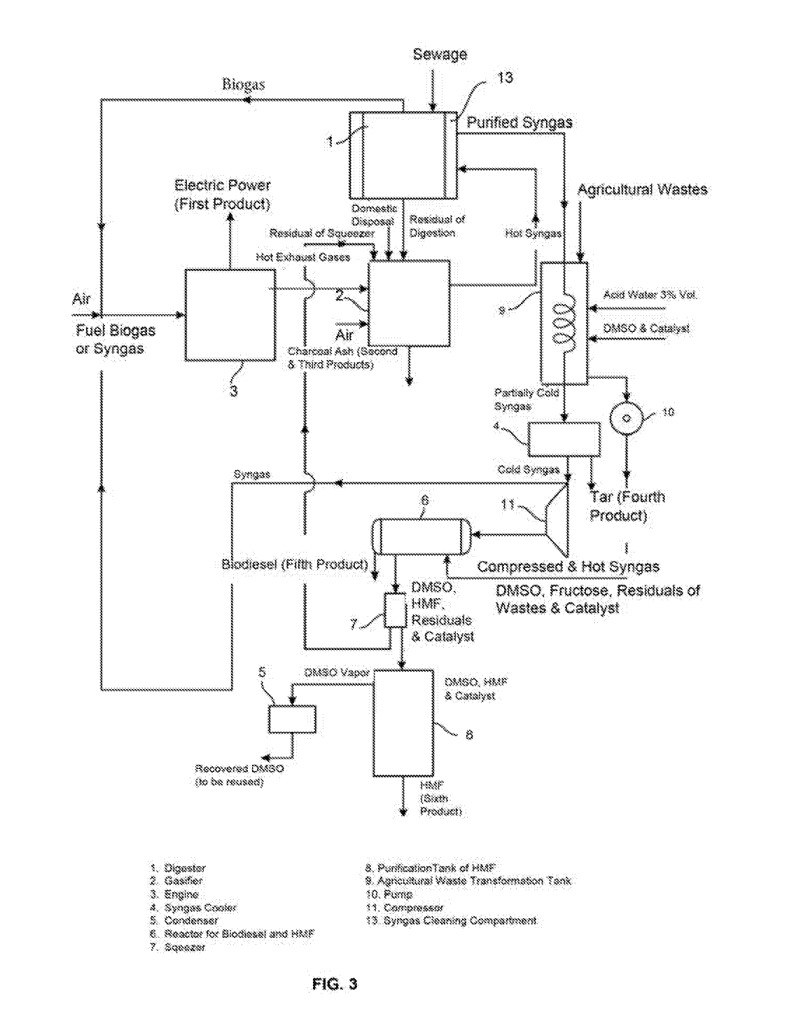

FIG. 3 depicts the substance flow scheme to confirm different types of methods as previously showed in FIG. 1 as follows:

The present device can be divided into four parts; the first which can be called “electrical power producer”, which comprises power generator 3, while the second part can be called “biogas producer” and comprises digester 1. The third part can be called “generator of biodiesel, active coal, tar and ashes” and comprises purification chamber 14, gas generator 2, radiator 4, compressor 11 and reactor 6. The fourth part is called “HMF Producer” and contains primary treatment tank 9, pump 10, reactor 6, extractor 7 and purification tank 8.

Referencing FIG. 3, the substances entering into the first part are air and fuel (biogas or syngas from the second and third parts, respectively) for the production of electrical power and hot exhaust gases. The substances entering into the second part are sewage waste and heat (from the third part) and acid solution (to adjust PH of digestion) for the production of biogas as fuel of the first part. The substances entering into the third part are air and exhaust gases for the conversion into gases (from the first part), household waste and silicon carbide, cobalt, calcium as a catalytic agent (for the Fischer-Tropsch process) and the rest of the second and fourth parts for the production of active coal, ashes and biodiesel. The fourth part receives heat (from compressed air in third part) and diluted aqueous acid solution 3%, DMSO as solvent, chromic chloride as a catalytic agent, and active coal to purify produced HMF.

As indicated in FIG. 3, for a complete understanding of the entire device, the colored paths shall be followed, as follows:

Black paths as main entries:
Sewage of digester 1
Household waste of gas generator 3
Remaining part from digester 1 to gas generator 3
Remaining part from extractor 7 to gas generator 3
Agricultural waste of primary treatment tank 9

Main products:
Electrical power (first product) from power generator 3
Active coal and ashes (second and third product respectively) from gas generator 3
Tar (fourth product) from radiator 4
Biodiesel (fifth product) from reactor 6
HMF (sixth product) from reactor 6

Orange paths of intermediary processes for gases:
Biogas is produced in digester 1 and then supplied to power generator 3 as fuel.
Hot exhaust gases are produced in power generator 3 and then supplied to gas generator 2 to convert the biomass into gases.
Syngas is generated during the biomass conversion into gases in gas generator 2. The gas is then supplied to chamber 13, not only to clean syngas from impurities in the presence of dolomite, eggshell, active coal, ashes and sand as catalytic agents, but also to heat up the digester solution to accelerate the digestion in digester 1.
Hot syngas is moved out from chamber 13 to be initially cooled during passage through primary treatment tank 9 through two types of liquids, which are diluted aqueous acid solution 3% and DMSO as solvent in the presence of 1% of mass of chromic chloride as a catalytic agent.
Initially cooled syngas is removed from primary treatment tank 9 to be cooled in radiator 4 to increase tar in syngas to prepare such gases for the compression process or use the same as fuel for power generator 3.
Cooled syngas is supplied to compressor 11 to be compressed to 25 bar. As a result of such compression, the temperature of syngas increases up to 300° C. to prepare the same for the Fischer-Tropsch process (conversion of syngas into liquid biodiesel in presence of a catalytic agent) in reactor 6.
Compressed syngas is supplied to reactor 6 at 25 bar and 300° C. in presence of 75% of silicon carbide—20% of cobalt—5% of calcium as a catalytic agent for the production of biodiesel.

Blue paths of intermediary processes for liquids:
Hot syngas is removed from chamber 13 for the purpose of heating primary treatment tank 9 to achieve the hydrolysis of agricultural wastes (conversion of cellulosic substances into glucose) in presence of diluted aqueous acid solution 3% at 180° C. for a period of four to six hours, and the conversion of produced glucose to fructose through strong flipping of such glucose with DMSO as solvent (1 liter of DMSO/0.8 kg of glucose) in presence of 1% of chromic chloride as a catalytic agent at 90° C. for one hour.
The mixture resulting from the previous process is composed of the rest of waste, DMSO, fructose and a catalytic agent. In addition, such mixture is removed from primary treatment tank 9 to reactor 6 by using pump 10 for the production of HMF through heating this mixture to 120° C. or 250° C. for two hours or fifteen minutes, respectively, in reactor 6, through compressed and hot syngas.
Mixtures previously produced in reactor 6 are consisting of the rest of the waste, DMSO, HMF and a catalytic agent, which is poured into extractor 7, to separate remaining agricultural wastes and generate solution from DMSO, HMF and a catalytic agent.
For the purification of HMF, the previous solution is moved to purification tank 8 which contains active coal to adsorb DMSO and, consequently, remove purified HMF from the bottom of such tank.
After obtaining DMSO from purification tank 8 with the presence of active coal and adsorbed DMSO, it can be heated up to 192° C. to evaporate DMSO and then supply resulting steam to radiator 5 to increase such steam and, as such, recapture DMSO for later use.

Digester 1 contains the sewage present in diluted acid and temperature of 35-45° C. of anaerobic digestion and the production of biogas after a digestion period of 15-20 days, which can be used as fuel for power generator 3. Power generator 3 produces electrical power being the first product of the device, and also produces exhaust that is used to supply gas generator 2 with heat and gases with oxygen. Gas generator 2 is supplied with household waste and the rest of digestion process, and the rest of the agricultural waste treatment process which is obtained from extractor 7. Gas generator 2 produces active coal and ashes as second and third products of this invention, and also produces syngas. Syngas is purified in chambers surrounding digester 1 to be heated, as such chambers contain dolomite, eggshell, charcoal, ashes, and sand to separate tar being the fourth product from this device. Meanwhile, purified syngas is cooled in two phases; the first phase is done in primary treatment tank 9 to convert the agricultural cellulosic wastes into glucose during the hydrolysis for 4 hours in diluted hydrochloric acid solution at 3%. The second phase of syngas cooling is done in radiator 4. About half the syngas after cooling is supplied to power generator 3, while the rest is compressed to 25 bar, heated to 300° C. through compressor 11 and then moved to reactor 6 in the presence of silicon carbide-calcium-cobalt as a catalyst for the conversion of syngas into biodiesel being the fifth product of this device. Furthermore, the glucose produced during the hydrolysis of agricultural cellulosic waste is dissolved by the addition of DMSO to the glucose in primary treatment tank 9 and stirring the mixture for an hour in the presence of chromic chloride to convert glucose into fructose. This mixture comprises of DMSO, glucose, fructose and chromic chloride. The unconverted biomass is pumped through primary treatment tank 9 to reactor 6 by pump 10 for the production of HMF being the sixth product through heating mixture at 120° C. or 250° C. for two hours or fifteen minutes, respectively, by the use of the above-mentioned compressed syngas. The resulting product, which is HMF, DMSO, chromic chloride and unconverted biomass, is first poured into extractor 7 to separate the remaining biomass and then the residue is supplied first to gas generator 2, and second to purification tank 8 filled with active coal to adsorb the solvent DMSO and extract the purified HMF being the sixth product of this invention. Purification tank 8 is heated to 192° C. to evaporate the solvent DMSO, and then such steam is intensified in radiator 5.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A device for producing energy from waste comprising:
a power generator;
a digester;
a biodiesel, active coal, tar, and ashes generator comprising a purification chamber, a gas generator, a radiator, a compressor, and a reactor;
an HMF producer comprising a primary treatment tank, a pump, a reactor, an extractor and a purification tank;
wherein the power generator may be at least one of a diesel motor, a gas turbine and steam cycle; and
wherein the digester is configured to produce biogas.

13. The device according to claim 1, wherein the digester comprises an assembly of twelve chambers.

14. The device according to claim 2 wherein the chambers comprise the purification path of syngas; and
wherein the chambers are filled with catalytic agents including dolomite in 4 chambers, eggshell in three chambers, active coal in two chambers, ashes in two chambers, and sand in one chamber; and
wherein the syngas is used in a heating digestion compartment.

15. The device according to claim 1 wherein hot exhaust gases, produced in the power generator, are supplied by the gas generator to convert biomass into gases.

16. The device according to claim 1 wherein hot syngas is configured to be removed from the chamber and cooled by a primary treatment tank comprising two types of liquids; and wherein the two types of liquids are diluted aqueous acid solution of 3% volume and DMSO; and wherein the DMSO is a solvent with 1% mass of a chromium chloride catalyst agent.

17. The device according to claim 1 wherein the device is configured to remove hot syngas from the chamber; and wherein the hot syngas is configured to heat the primary treatment tank to accomplish hydrolysis for agricultural waste; and wherein the hydrolysis may comprise diluted aqueous acid solution of 3% volume at 180° C. for four to six hours; and wherein the device is configured to convert produced glucose into fructose by stirring the glucose with DMSO; and wherein the DMSO is configured as a solvent comprising 1 liter of DMSO/0.8 kg glucose with 1% of chromium chloride as a catalyst agent.

18. The device according to any of claims 1 wherein the device is configured to remove the initially cooled syngas from the primary treatment tank for cooling in the radiator; and wherein the device is configured to cool the syngas to condense tar therefrom and process it as fuel for the power generator.

19. The device according to claim 1 wherein the device is configured to move cooled syngas to the compressor for compressing it to 25 bar thereby increasing its temperature to 300° C.; and wherein the device is configured to prepare the syngas for a Fischer-Tropsch process; and wherein the device is configured to move the compressed syngas to the reactor at 25 bar and 300° C. with 75% silicon carbide, 20% cobalt, and 5% calcium as a catalyst agent.

20. The device according to claim 1 wherein the device is configured to produce a mixture comprising the remaining waste, DMSO, fructose, and catalyst agent; and wherein the remaining waste, DMSO, fructose, and catalyst agent is removed from the primary treatment tank to the reactor by using a pump; and wherein the device is configured to produce HMF by heating the mixture to 120° C. or 250° C. in the reactor for at least one of 2 hours and fifteen minutes respectively.

21. The device according to claim 9 wherein the device is configured to move the solution to the purification tank which contains active coal for adsorption of DMSO; and wherein the device is configured to subsequently filter LHMF from the bottom of the purification tank with an absorption ratio of active coal to DMSO of 10 g:50 ml.

22. The device according to claim 10 wherein the device is configured to heat the purification tank to 192° C. for the evaporation of DMSO after the extraction of DMSO from the purification tank; and wherein the device is configured to move the resulting steam to the radiator to intensify the steam for recaptured DMSO for later use.
